(19) United States
(12) Patent Application Publication
(10) Pub. No.: US 2024/0325292 A1
(43) Pub. Date: Oct. 3, 2024

(54) COMPOSITION AND METHODS OF TREATMENT USING TRANSDERMAL SUPPLEMENTATION

(71) Applicant: Xygenyx Inc., Pleasant Hill, CA (US)

(72) Inventor: Alexy Goldstein, LaFayette, CA (US)

(21) Appl. No.: 18/295,188

(22) Filed: Apr. 3, 2023

(51) Int. Cl. A61K 9/00; A61K 31/33; A61K 31/355; A61K 31/357; A61K 35/74; A61K 36/28; A61K 36/886

(52) U.S. Cl. CPC A61K 9/0014; A61K 31/33; A61K 31/355; A61K 31/357; A61K 35/74; A61K 36/28; A61K 36/886

(57) ABSTRACT

A composition comprising an aqueous gel with therapeutically effective amounts of carnosine, vitamin E, aloe vera gel, and a Silybum marianum extract. The extract of the Silybum marianum plants may be silymarin, and the addition of a therapeutically effective amount of a Lactobacillus ferment lysate may also be used.

### CROSS REFERENCE TO RELATED APPLICATIONS

Priority is claimed in the application data sheet to the following patents or patent applications, each of which is expressly incorporated herein by reference in its entirety:
None.

### BACKGROUND OF THE INVENTION

### Field of the Art

The disclosure relates to the field of hormone supplementation therapies, and more particularly to the provision of a homeopathic gel for topical treatment of humans with therapeutically-beneficial additional compounds.

### Discussion of the State of the Art

Protecting the skin can help reduce the risk of long-term degenerative effects such as excessive dryness, skin cancer, hyperpigmentation, and age-related sign such as wrinkles or loss of skin firmness or texture. Many skincare products exist which attempt to address these concerns through topical applications of UV-blocking agents and oils, however they fail to address the underlying causes of these effects and merely treat the symptoms while attempting to prevent further damage. To adequately prevent and repair skin damage, a product needs to stop oxidative stress and glycation of the affected cells, and neutralize the products of these processes to prevent further damage.

In addition, there are multiple skin types that require different approaches as they differ in their reaction to environmental factors, absorption and permeability, and the rates and levels of these damaging processes and cellular products that must be addressed. To optimally protect and restore the skin, products must be tailored to individual with active melanogenesis or higher skin pigment production, those with little pigmentation, those with thicker or tougher skin that may be more difficult for compounds to adequately penetrate for absorption, and those with sensitive or delicate skin that is more easily damaged.

What is needed is a means of providing transdermal skincare supplementation, that uses synergistic combinations of active ingredients to improve skin protection during daylight hours for users with light or low melanin, or sensitive or damaged skin.

### SUMMARY OF THE INVENTION

Accordingly, the inventors have conceived and reduced to practice a composition and methods of treatment using transdermal supplementation.

According to a preferred embodiment, a composition for transdermal hormone supplementation is disclosed, comprising an aqueous gel with therapeutically effective amounts of carnosine, vitamin E, aloe vera gel, and a Silybum marianum extract. The extract of the Silybum marianum plants may be silymarin, and the addition of a therapeutically effective amount of a Lactobacillus ferment lysate may also be used.

According to another preferred embodiment, a method of treatment using transdermal supplementation, comprising the step of: applying an aqueous gel to a human's skin from a dispenser, the aqueous gel comprising: carnosine; aloe vera gel; vitamin E; and an extract of the Silybum marianum plant. The extract of the Silybum marianum plants may be silymarin, and the addition of a therapeutically effective amount of a Lactobacillus ferment lysate may also be used.

### DETAILED DESCRIPTION

Headings of sections provided in this patent application and the title of this patent application are for convenience only and are not to be taken as limiting the disclosure in any way.

The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

Techniques and mechanisms described or referenced herein will sometimes be described in singular form for clarity. However, it should be appreciated that particular aspects may include multiple iterations of a technique or multiple instantiations of a mechanism unless noted otherwise.

Human Growth Hormone (HGH), also known as somatropin, is a hormone comprising a peptide chain that is synthesized in the pituitary gland and is present throughout the body in varying concentrations. HGH stimulates growth and cell reproduction, as well as cell regeneration. HGH stimulates production of insulin-like growth factor 1 (IGF-1), which is a key hormone in regulating growth in children; it also has anabolic effects in adults and thus is commonly used in conjunction with anabolic steroids in stimulating muscle and bulk growth in athletes. HGH has a short biological half-life (of 10-20 minutes), and so is being continuously synthesized in the human body as needed, regulated by a complex web of regulatory interactions between hormones and various metabolites.

Somatropin is a recombinant analogue of HGH or somatotropin, which is available as a prescription drug to treat various growth hormone disorders. It is also used increasingly in vitality therapies for elderly patients, through off-label prescriptions. Similarly, somatropin has been used for decades by competitors in sports because of its anabolic properties.

Tocopherols are a group of four organic compounds collectively known as vitamin E, occurring in alpha (α), beta (β), gamma (γ), and delta (δ) forms each comprising a chromane ring and a hydroxyl group, the particular form being determined by the methylation of the chromane ring. These forms vary slightly in their chemical structure and biological activity, as shown and described below. All tocopherols are potent inhibitors of reactive oxygen species at a cellular level, reducing cancer risks, cellular aging, and the development of cardiovascular disease. When used in topical applications in conjunction with carnosine, the mixture has synergistic benefits for skin health, improving firmness, smoothness, and hydration as well as reducing the depth of various wrinkles by 24-27% after just one month of regular application.

α-tocopherol, the chemical structure of which is shown below, is the most common and biologically active form and is commonly used in supplements with dosages measured in International Units (IU). It is a potent antioxidant that helps to protect cell membranes and other lipids from oxidative damage. Alpha-tocopherol is primarily found in foods of animal origin, such as liver, egg yolk, and dairy products. In large doses of 1600-3200 IU/day, the antioxidant effects of α-tocopherol significantly lower blood plasma concentration of F2-isoprostane. Dietary supplementation of α-tocopherol in doses up to 400 IU/day has also been shown to reduce the risk of developing neurodegenerative diseases such as Alzheimer's Syndrome or dementia by as much as 24% on controlled trials, as well as reducing the risk of coronary disease by up to 34% and contributing to lower risk of developing a number of cancers including kidney, lung, and bladder cancers. In combination with 10-20 mg β-carotene and 400-500 mg vitamin C, α-tocopherol has a synergistic effect that has been shown to dramatically reduce the progression of age-related macular degeneration in doses around 400-500IU, as well as a 23% reduction in the development of age-related cataracts. α-tocopherol has also been shown to improve functional health of sperm in doses of 800-900 IU/day, with additional synergistic benefits in combination with vitamin C.

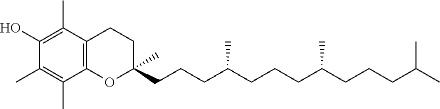 [SMILES: Cc1c(C)c2c(c(C)c1O)CC[C@@](C)(CCC[C@H](C)CCC[C@H](C)CCCC(C)C)O2]

β-tocopherol, the chemical structure of which is shown below, is also a potent antioxidant but is less biologically-active than the α-tocopherol form. It is found in plant-based foods, such as soybeans, nuts, and seeds as well as in various oils such as soybean, grapeseed, and olive oils. The relative biological activity of β-tocopherol is 0.5 as compared to α-tocopherol (i.e., assuming α-tocopherol has a biological activity factor of 1.0).

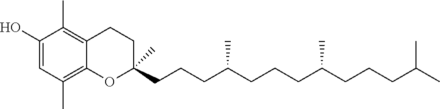 [SMILES: Cc1cc(O)c(C)c2c1O[C@](C)(CCC[C@H](C)CCC[C@H](C)CCCC(C)C)CC2]

γ-tocopherol, the chemical structure of which is shown below, is the second-most abundant form after α-tocopherol and has similar antioxidative capacity. It is found in high concentrations in nuts, seeds, and vegetable oils. It is a more effective scavenger of certain types of free radicals than alpha-tocopherol and is believed to play a key role in reducing inflammation and promoting heart health. However, due to the preferential uptake of the α-tocopherol form, γ-tocopherol is found at roughly 10% of the concentration of the preferred variant in vivo, even when dietary intake occurs in equal portions. The relative biological activity of the γ-tocopherol form is 0.1.

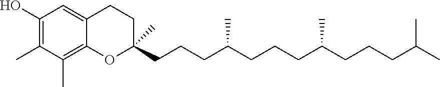 [SMILES: Cc1c(O)cc2c(c1C)O[C@](C)(CCC[C@H](C)CCC[C@H](C)CCCC(C)C)CC2]

δ-tocopherol, the chemical structure of which is shown below, is found in small amounts in some plant-based foods and has a relative biological activity of only 0.03. It has been shown to have antioxidant properties, like other forms of vitamin E. It can help protect cell membranes and other lipids from oxidative damage caused by free radicals. Additionally, it has been suggested that δ-tocopherol may play a role in preventing the formation of harmful nitrogen-containing compounds, which can contribute to the development of certain diseases. Some studies have also suggested that δ-tocopherol may have anti-inflammatory properties. In one study, for example, supplementation with δ-tocopherol reduced markers of inflammation in the blood of individuals with metabolic syndrome. δ-tocopherol has also been suggested to play a role in immune system function.

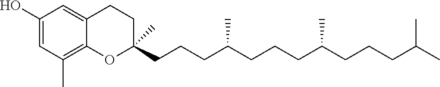 [SMILES: Cc1cc(O)cc2c1O[C@](C)(CCC[C@H](C)CCC[C@H](C)CCCC(C)C)CC2]

Tocotrienols are another group belonging to the vitamin E family, differing in structure from their analogous tocopherol forms by the presence of three double-bonds in the hydrophobic side chain. Like tocopherols, tocotrienols have antioxidant properties that help to protect cells from oxidative stress and damage caused by free radicals. Tocotrienols have been shown to support cardiovascular health by helping to lower cholesterol levels and improve blood flow. They have also been suggested to have anti-inflammatory properties, which may further benefit heart health. Tocotrienols may play a role in protecting the brain from damage and degeneration. Studies have suggested that they may help to improve cognitive function, reduce the risk of stroke, and protect against neurodegenerative diseases like Alzheimer's. Tocotrienols have been shown to have anti-cancer properties in laboratory studies. They may help to prevent the growth and spread of cancer cells, and may also enhance the effects of chemotherapy and radiation therapy.

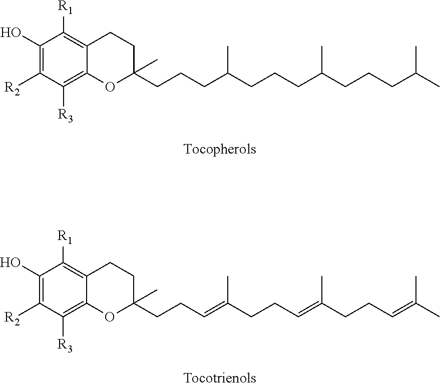

Aloe vera (also known as Aloe barbadensis, Aloe indica, Aloe vulgaris, and a number of other common-use names) is a succulent of the genus Aloe, widely cultivated for a variety of medicinal and cosmetic uses and one of the oldest known medicinal plants. Many of aloe's therapeutic properties are attributed to the phenols and polysaccharides in the gel of its leaves, 5 which comprises roughly 98.5-99.5% water with the balance comprising over 200 known compounds in varying portions. Many of the primary medicinal compounds found in aloe are mucopolysaccharides (MPS) characterized by long-chain sugars as shown below.

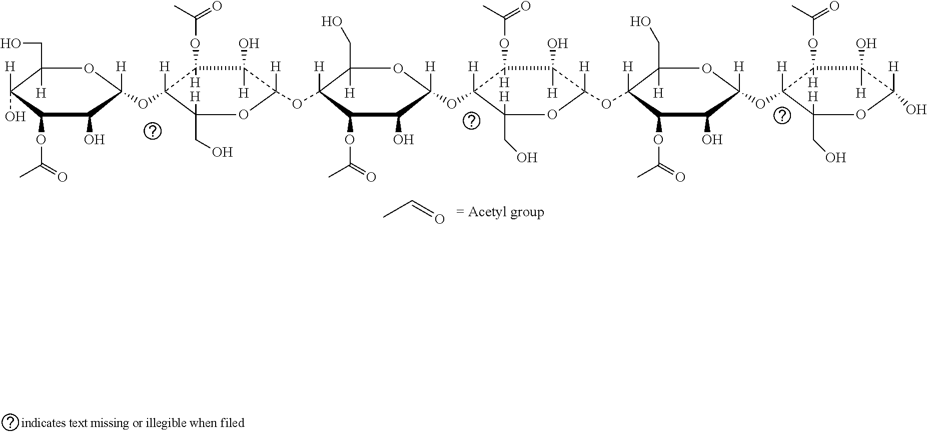

The aloe gel also contains mono/oligosaccharides and hemicelluloses which make up the more inert fraction of the phytocomplex shown below. Other compounds present include a variety of sugars, glycoproteins, flavonoids, minerals, enzymes, and various amino acids.

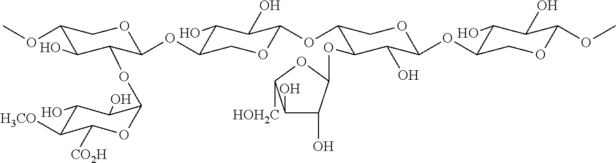

Aloe gel is commonly used in a variety of topical applications with therapeutic benefits for skin health and healing of minor injuries such as abrasions, burns, rashes, psoriasis, cold sores, or dry skin and is often combined with antioxidants such as vitamin E for synergistic benefits. For increased benefit, aloe gel may be enzymatically treated to hydrolyze hemicelluloses (thus transforming them into additional sugars and saccharides), or it may be fermented to boost the concentrations of various phytocomplexes present, resulting in as much as a fourfold increase in antioxidative capacity of the gel. In particular, fermenting aloe gel doubles both the amino acid and polyphenol content present in the gel, dramatically improving their availability for therapeutic uses. Aloe vera gel can help to enhance the absorption and efficacy of other beneficial ingredients, such as vitamin E, vitamin C, or hyaluronic acid. Look for skincare products that contain aloe vera gel along with other active ingredients. Aloe vera gel can be combined with other natural ingredients, such as honey, coconut oil, or tea tree oil, to create a nourishing and healing skincare product. These ingredients can help to enhance the moisturizing, antibacterial, or anti-inflammatory properties of aloe vera gel.

Silybum marianum, known by various common names including milk thistle, Scotch thistle, and Saint Mary's thistle, is a plant of the family Asteraceae originally native to southern Europe but now found throughout the world. Various plant parts and extracts of milk thistle have been used in traditional medicine in various cultures for centuries, in particular the extract silymarin, a polyphenol which comprises the compounds silibinin A and B, the chemical structures of which are shown below (silibinin A at left, silibinin B at right), and which is typically used in concentrations of 0.1-5% in various topical formulations. Silymarin works in conjunction with other free radical scavengers such as glutathione and tocopherols, improving the capture of free radicals and reducing oxidative stress. Silymarin has also been shown to specifically reduce the process of lipid peroxidation, protecting lipids against oxidative damage that inhibits the uptake of fat-soluble compounds, making for a potent synergy with tocopherols that boosts their efficacy as antioxidants while also improving their bioavailability. When applied topically, silymarin has also been shown to have anti-inflammatory properties and dramatically reduces the occurrence of acne lesions in daily therapeutic doses around 210 mg.

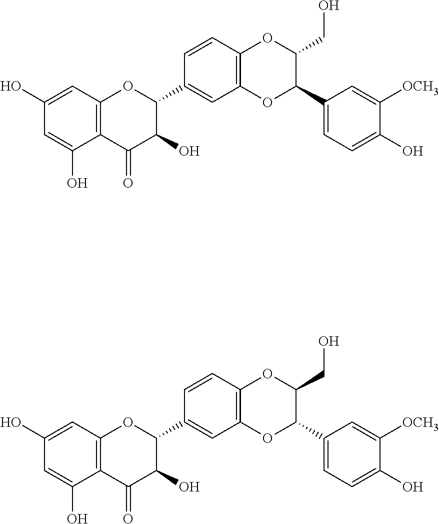 [SMILES: COc1cc([C@H]2Oc3cc([C@H]4Oc5cc(O)cc(O)c5C(=O)[C@@H]4O)ccc3O[C@@H]2CO)ccc1O ; COc1cc([C@@H]2Oc3cc([C@H]4Oc5cc(O)cc(O)c5C(=O)[C@@H]4O)ccc3O[C@H]2CO)ccc1O]

Carnosine, the chemical structure of which is shown below, is a dipeptide of beta-alanine and histidine, found exclusively in meats and other animal-based dietary sources (there are no known plant-based dietary sources for carnosine). When used topically in concentrations generally ranging from 0.1-2% (though sometimes as high as 5% or more, depending on the method of application and intended target benefits), carnosine has a number of antioxidant properties including both scavenging free radicals as well as unsaturated aldehydes produced by cellular lipid membranes during oxidative stress via the process of lipid auto-peroxidation. It also has anti-glycation properties, reducing the formation of glycation end-products that contribute to premature aging effects and a variety of degenerative disorders such as Alzheimer's and diabetes. Other benefits of carnosine include buffering the pH of muscle tissue, functioning as a neurotransmitter (high levels of carnosine are found in neural tissue), and slowing a number of aging processes by increasing the Hayflick limit (the programmed limit on the number of times a cell population will divide before cell division ceases) in cells and slowing the telomere shortening rate. These properties make carnosine a useful adjunct in combination with silymarin and tocopherols, where their respective antioxidant and anti-aging benefits work synergistically to complement each other. In test studies, topical applications of this synergistic mixture have been shown to improve skin health and reduce age-related wrinkles by as much as 27%.

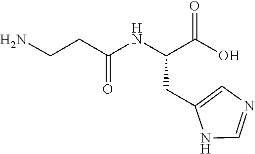 [SMILES: NCCC(=O)N[C@@H](Cc1cnc[nH]1)C(=O)O]

Lactobacillus is a genus of rod-shaped bacteria common to the gastrointestinal tract, many of which are marketed as a probiotic supplement in a number of food products such as milk, yogurt, cheese, kefir, and other dairy products. Lactobacillus species are commonly used in fermented dairy and other food products such as sauerkraut, where they break down lactose and other sugars and produce lactic acid to impart a “tart” flavor characteristic of fermented foods. In topical applications, Lactobacillus may themselves be fermented to produce a nonliving lysate, which has been shown (in topical applications such as serums, creams, or sprays, and typically in concentrations between 0.5-5%) to provide a soothing effect and strengthen the skin's ability to function as a defensive layer against pathogens. This is due to encouragement of the microbiota living on the skin, where the Lactobacillus ferment lysate helps maintain an ideal mix of flora to reduce the occurrence and severity of a number of skin conditions caused by dysfunction of the skin barrier. The lysate inhibits the reproduction of harmful bacteria on the skin to regulate the population, and also regulates the production of skin oils to reduce irritation from under- or over-production.

Coffea canephora is a species of coffee plant originally native to sub-Saharan Africa, also commonly known as Coffea robusta. In addition to being brewed as coffee (often roasted), the seeds of this plant contain a variety of compounds with therapeutic uses that can be extracted and used in formulations for internal or topical use, including antioxidants, alkaloids, amino acids, and volatile compounds. Extracts from green coffee beans (dried, un-roasted beans with the husks removed) have proven beneficial in skin care for protection from harmful effects of sun exposure, reducing cellulite formation, and improving complexion both in skin and hair care. Coffee bean extract has been shown to increase collagen production, improving skin firmness, hydration, and texture and contributing to overall skin health. The extract also acts as a diuretic, further contributing to skin benefits particularly in improving firmness and reducing the appearance of wrinkles. Typical concentrations vary widely due to the breadth of applications and the large number of active compounds within the extracts, but often remain below 5% concentration in topical applications.

Citric acid, the chemical structure of which is shown below, is a weak organic acid occurring naturally in a wide variety of fruits, notably those of the citrus family with lemons and limes having the highest naturally-occurring concentrations (as high as 8% by dry weight). It is commonly used as a food additive to impart a sour or “tart” flavor as an acidulant, as well as in various creams, gels, and liquids for cosmetic or topical medicinal purposes. It has astringent, exfoliating, and antioxidant properties, which make it a popular ingredient in a variety of topical applications; it is often used as a natural exfoliant in skincare products at a concentration of 5-10%, such as facial scrubs and body washes. Its acidic properties help to slough away dead skin cells and unclog pores, leaving the skin looking smoother and more radiant. Citric acid can help to brighten the skin when used in lower concentrations of 1-5% and reduce the appearance of age spots, sun damage, and other types of hyperpigmentation by inhibiting the production of melanin, the pigment that gives skin its color. Citric acid has antioxidant properties that can help to protect the skin from damage caused by free radicals, which can lead to premature aging. It is often used in anti-aging skincare products to help reduce the appearance of fine lines and wrinkles, and has a synergistic effect when combined with vitamin E and aloe gel. Citric acid can help to control excess oil production in the skin, which is a common cause of acne. It can also help to unclog pores and reduce inflammation, making it a popular ingredient in acne treatment products.

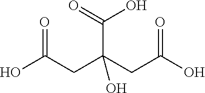 [SMILES: O=C(O)CC(O)(CC(=O)O)C(=O)O]

Allantoin, the chemical structure of which is shown below, is a diureide of glyoxylic acid, produced from uric acid during purine catabolismit may also be extracted from the leaves of the comfrey plant; comfrey leaves have been used for centuries to aid in skin healing and reducing swelling around minor injuries. While allantoin is also found in a number of plants including beets, chamomile, and tobacco seeds, therapeutic usage often relies on synthetically-produced allantoin for practical reasons and applied topically in concentrations up to 2%. Allantoin is a natural humectant, helping to draw moisture to the skin and prevent dryness to improve skin hydration and leave the skin feeling soft and smooth. Allantoin has anti-inflammatory properties that can help to soothe and calm irritated or inflamed skin. It is often used in products for sensitive skin or skin conditions such as eczema or psoriasis, and it has been shown to enhance the healing of wounds and promote the growth of new tissue. It can help to improve skin elasticity and reduce the appearance of scars and stretch marks. Allantoin also has antioxidant properties that can help to protect the skin from damage caused by free radicals, which can lead to premature aging. It can help to reduce the appearance of fine lines and wrinkles and improve skin firmness and elasticity. These properties make it a useful agent in combination with aloe vera gel for improving skin health and reducing the long-term effects of oxidative damage.

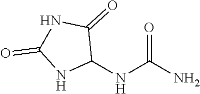 [SMILES: NC(=O)NC1NC(=O)NC1=O]

Dextran, the chemical structure of which is shown below, is a branched polysaccharide polymer originally isolated from wine and commonly produced at an industrial scale using bacterial cultures. Dextran has a number of medical uses and is used in varying concentrations as high as 5% depending on the administration method and intended uses, which include use as a blood plasma expander, an anti-coagulant, use in scaffold material for tissue engineering (such as during reconstructive surgery or for growing grafts for later implantation), as well as immunomodulation making it a useful adjunct in vaccines. Dextran is also a useful drug delivery vehicle, where it is used in combination with various compounds and targeted to specific tissues or cells in the body. This can increase the efficacy and reduce the toxicity of certain drugs, making it beneficial in combination with many ingredients for enhancing their respective effects.

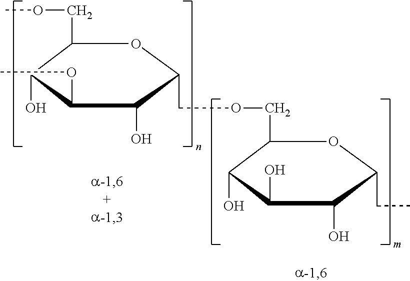

Nonapeptide-1 (also known under the trademarked name MELANOSTATINE™ 5), the chemical structure of which is shown below, is a peptide consisting of nine amino acids linked together by peptide bonds. Peptides are small chains of amino acids that play important roles in many biological processes in the body, including cell signaling, enzyme regulation, and structural support. Nonapeptides can have a variety of functions depending on their specific sequence and context. For example, some nonapeptides are involved in regulating the activity of hormones, neurotransmitters, or growth factors. Others can act as antimicrobial agents or immune modulators. One well-known nonapeptide is oxytocin, which is sometimes called the “love hormone” or “cuddle hormone” because it is released in the brain during social bonding activities such as hugging, kissing, or breastfeeding. Oxytocin also plays a role in childbirth, lactation, and sexual reproduction. Nonapeptides can be synthesized in the laboratory or isolated from natural sources such as plants, animals, or microorganisms. They have a wide range of potential applications in medicine, biotechnology, and materials science, including drug discovery, tissue engineering, and biosensors, and are often used in concentrations as high as 1-2%.

Nonapeptide-1 in particular is a synthetic peptide that is used in the cosmetic industry for its skin brightening properties. Nonapeptide-1 works by inhibiting the production of melanin, the pigment that gives skin its color. Melanin production is controlled by a number of different enzymes, and one of the key enzymes involved in this process is called tyrosinase. Nonapeptide-1 works by blocking the activity of tyrosinase, which in turn reduces the amount of melanin produced by the skin. This can help to even out skin tone and reduce the appearance of age spots, sun damage, and other types of hyperpigmentation. In addition to its skin brightening properties, nonapeptide-1 has been shown to have antioxidant and anti-inflammatory effects. These properties can help to protect the skin from damage caused by free radicals and reduce inflammation, which can contribute to skin aging.

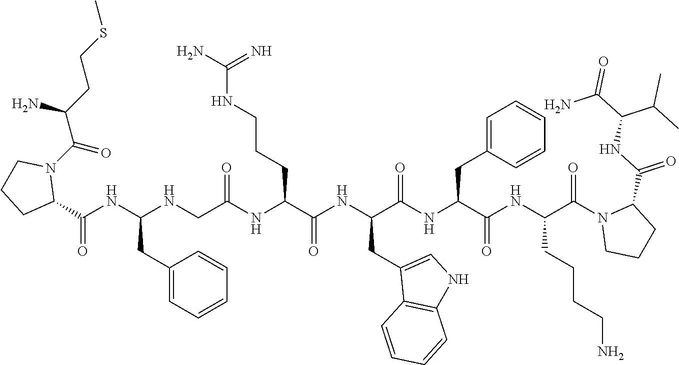 [SMILES: CSCC[C@H](N)C(=O)N1CCC[C@H]1C(=O)N[C@H](Cc1ccccc1)NCC(=O)N[C@@H](CCCNC(=N)N)C(=O)N[C@H](Cc1c[nH]c2ccccc12)C(=O)N[C@@H](Cc1ccccc1)C(=O)N[C@@H](CCCCN)C(=O)N1CCC[C@H]1C(=O)N[C@H](C(N)=O)C(C)C]

Dextran and nonapeptide-1 can work together in different ways, depending on the specific context or application. In some cases, dextran and nonapeptide can be used to improve the delivery of drugs to specific cells or tissues in the body. Dextran is a type of polysaccharide that can form a hydrogel or gel-like structure when mixed with water. This structure can be used to encapsulate drugs and protect them from degradation or elimination in the body. Nonapeptide-1, on the other hand, can bind specifically to certain types of cells or tissues, such as cancer cells or inflamed tissues. By attaching nonapeptide to the surface of dextran-based drug carriers, the carriers can be targeted to specific cells or tissues, increasing the efficacy and reducing the side effects of the drugs.

Dextran and nonapeptide can also be used together to promote wound healing. Dextran hydrogels have been shown to have excellent wound-healing properties, including the ability to absorb excess exudate (fluid) and create a moist environment that promotes tissue regeneration. Nonapeptide can stimulate the proliferation and migration of certain types of cells, including fibroblasts and endothelial cells, which are important for tissue repair and angiogenesis (the formation of new blood vessels). By combining dextran and nonapeptide, a wound dressing or scaffold can be created that enhances the natural healing processes of the body and accelerates the closure of the wound.

Niacinamide (also known as nicotinamide), the chemical structure of which is shown below, is a form of vitamin B3 that occurs in trace amounts mainly in meat, fish, nuts, and mushrooms, as well as to a lesser extent in some vegetables. It has been shown to have anti-inflammatory and sebum-regulating properties in topical applications with concentrations typically ranging between 2-10%, which can be helpful in the treatment of acne and can reduce redness and inflammation associated with rosacea. It can help to reduce the production of sebum, which can contribute to acne, and also reduce inflammation and redness. Niacinamide also reduces the appearance of hyperpigmentation, including age spots and melasma, by inhibiting the transfer of melanin from melanocytes to the surrounding skin cells. Niacinamide has been shown to improve the appearance of fine lines and wrinkles, and can also help to improve skin elasticity and texture by increasing the production of collagen and other proteins that are important for skin health. Niacinamide has also been shown to have photoprotective properties, which can help to reduce the risk of skin cancer by reducing DNA damage caused by UV radiation and by reducing the production of free radicals. Niacinamide also increases the biosynthesis of ceramides in the skin, strengthening the skin as a permeability barrier.

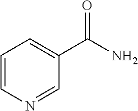

Sodium hyaluronate, the chemical structure of which is shown below, is the sodium salt of hyaluronic acid, a key component in connective, neural, and epithelial tissues as well as synovial fluid and extracellular matrix in the human body. It functions as a tissue lubricant by forming a viscoelastic solution in water, modulating the interactions of various tissues as they move against each other. It is therefore commonly used in dietary supplements to promote joint and skin health, often in combination with collagen. In gel form, sodium hyaluronate forms a cross-linked polymer that can be used to envelop and deliver other compounds for absorption, enabling topically-applied sodium hyaluronate to help facilitate the absorption of macromolecules such as HGH or somatropin, without the need for invasive delivery methods. This functions through the process of endocytosis, a cellular process in which cells take in materials by engulfing them with their cell membrane. When sodium hyaluronate is applied topically to the skin or mucous membranes, it can interact with the cells in these tissues, stimulating endocytosis and allowing for improved absorption and greater bioavailability of larger molecules. The concentration of cross-linked polymer sodium hyaluronate (also known as cross-linked hyaluronic acid or cross-linked HA) can vary depending on the specific formulation of the product. However, in topical skincare products, the concentration of cross-linked polymer sodium hyaluronate is typically between 0.1% and 2%.

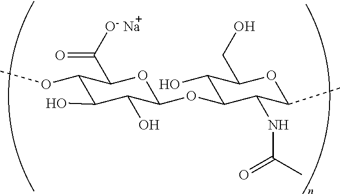

Squalane, the chemical structure of which is shown below, is a hydrogenated derivative of squalene, a naturally-occurring oil that is secreted by the skin particularly in regions of the forehead, nose, and ears. Squalane is commonly sourced from shark liver oil, as well as plant-based sources including olive oil, rice, and sugar cane, and is used in topical applications in concentrations as high as 5%. The complete hydrogenation of squalane improves its resistance to oxidative damage, making it more stable and thus more suitable for use in therapeutic products as it is not subject to auto-oxidation resulting in gradual loss of efficacy. This also gives squalane a higher molecular weight and contributes to a less “greasy” feel when used in topical medications or cosmetics, and it is readily absorbed into the skin reducing the sensation of residue on the surface.

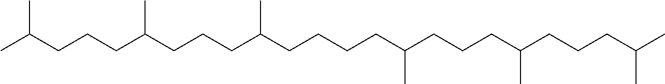 [SMILES: CC(C)CCCC(C)CCCC(C)CCCCC(C)CCCC(C)CCCC(C)C]

At a cellular level, squalane strengthens the lipid barrier layer of cells, promoting proper membrane function in the absorption and bioregulation of compounds and improving the intracellular production of ATP. It is also an intermediate compound in the production of sterol, the building block of a variety of biologically-important compounds including cholesterols and steroids. The chemical structure of sterol is shown below.

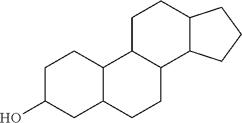 [SMILES: OC1CCC2C(CCC3C4CCCC4CCC23)C1]

Delivery of large biomolecules (such as HGH) or biosimilars (such as somatropin) has largely been limited to invasive routes (e.g., intramuscular and subcutaneous injection), because of their poor absorption and enzymatic degradation. However, administration via invasive routes presents several disadvantages—pain and risk of infection from syringes, unsuitability for administration to “needle-phobic” patients, and difficulty in providing effective self-administration. But delivering such medicaments transdermally is challenging because of the large size and complex electrochemistry of the molecules and the effectiveness of the skin barrier; in particular, the stratum corneum, which is the outermost part of the skin. Because of the stratum corneum, only low-molecular weight drugs with moderate lipophilicity are typically transferred administered transdermally via passive diffusion (for example, via patches or gels). Mechanical or other technological aids to skin barrier evasion, including iontophoresis or microneedles, provide some help, but these require medical professionals and are expensive and difficult to administer over time. A more advantageous approach would be to use penetration (or permeation) enhancers to help large biomolecules across the skin barrier without technological assistance. Even more preferable would be the use of natural phytochemicals to act as penetration enhancers to help large biomolecules cross the skin. Additionally, it is advantageous in some cases to administer a smaller precursor molecule that more easily crosses the skin barrier and then stimulates synthesis of the larger target biomolecules in vivo.

For example, L-DOPA is an amino acid that is synthesized in vivo in humans; it is a precursor to the crucial neurotransmitters dopamine, norepinephrine, and epinephrine. It has psychoactive properties and is used as a treatment in Parkinson's disease. L-DOPA has also been shown in numerous studies to promote the increase of serum HGH levels in humans. L-DOPA is a key phytochemical in velvet beans (Mucuna pruriens). The chemical formula of L-DOPA is:

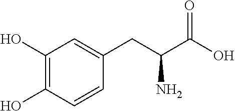 [SMILES: N[C@@H](Cc1ccc(O)c(O)c1)C(=O)O]

L-DOPA can be transdermally administered, preferably as a naturally-occurring phytochemical (such as via velvet bean extract), to supplement systemic HGH levels by providing increased precursor concentrations.

Another unconventional approach to transdermally administering large biomolecules is via the use of homeopathic principles. It has been shown that the use of a homeopathic preparation of somatropin-which does not require a prescription—can provide therapeutic benefits when delivered transdermally. If one assumes that homeopathic principles have validity, then the use of highly-diluted and activated formulations may provide a like benefit to that provided by injection of prescribed large doses of the same medicament. For example, over more than ten years a gel that includes a homeopathic formulation of somatropin at 30D dilution provides real benefits to its users, including an increase in serum HGH levels after application of the gel. It is likely that the fact that homeopathy does not rely on the actual presence of large amounts of the target biomolecule, but rather it relies on the presence of an energetically-activated aqueous solution of small phytochemicals. Moreover, a high degree of synergy is achieved by using natural photochemical penetration enhancers to improve the transdermal delivery rate of the energized homeopathic formulation. Thus the use of a variety of means to overcome the intrinsic difficulties of transdermal hormone supplementation provides the benefits of such supplementation without the risks of injecting prescription-based hormone treatments.

Another rapidly emerging area of natural health treatments is based on exploiting the rich phytochemical landscape made available by the opening up of legal cannabis products. Cannabis has been extensively cultivated throughout the world for centuries. Worldwide effects-targeted cultivation has progressed to the point where there are hundreds, if not thousands, of cultivars or strains which contain differing levels of the many active compounds that give cannabis its wide range of sought-after medical effects. One family of active compounds that are specific to cannabis is the cannabinoid family, of which more than 80 have been identified that may have overlapping but different medical effect profiles. These have been further separated into subclasses comprising: tetrahydrocannabinols (THC); cannabidiols (CBD); cannabichromenes (CBC); cannabinol (CBN); cannabigerols (CBG); cannabinodiol (CBDL); and other cannabinoids (cannabicyclol (CBL), cannabielsoin (CBE), cannabitriol (CBT) and other miscellaneous types.

In raw cannabis, the cannabinoids within the flowers' trichomes are carboxylated, meaning they have a COOH carboxylic acid group in their structure. These are mildly, if at all, psychoactive. In order for there to be psychoactivity the cannabinoids must be decarboxylated, or “decarbed” in layman's terms. Non-psychoactive THCA, for example, is decarboxylated to THC, a psychoactive cannabinoid. Both drying (or aging) and heat accomplish this chemical reaction, but the application of heat also results in the loss of certain other cannabis components such as the terpenes, which are highly volatile. Both the decarboxylated cannabinoids and the carboxylated cannabinoids exhibit important therapeutic effects for health, and although one can be chemically converted into the other, carboxylated cannabinoids becoming decarboxylated cannabinoids, As used herein both will be referred to as simply cannabinoids.

The biological effects of the cannabinoids may be mediated through two receptors specific for a class of endogenous cell signaling molecules, such as but not limited to N-arachidonoylethanolamine also known as AEA or anandamide, a neurotransmitter that predominantly binds to the cannabinoid receptor CB1 and a second compound, 2-arachidonoylglycerol also known as 2-AG which predominantly binds to the CB2 receptor. Expression of the CB1 receptor is found at highest levels in the central nervous system of humans and animals, particularly in the hippocampus, basal ganglia, neocortex, and spine (although expression in peripheral organs such as but not limited to the peripheral nervous system, liver, intestines, and kidneys is also present). Activation of the CB1 receptor has been linked to feelings of euphoria and an increase in appetite, among other effects. Expression of the CB2 receptor is found in the central and peripheral nervous system, the immune system and immune response-related cells, and peripheral organs, among other areas of expression. Activation of the CB2 receptor may have an analgesic effect, reduce inflammation, and increase of immune response towards certain pathogenic bacteria and fungi. The CB2 receptor may also mediate anti-cancer effects attributed to cannabinoids. Most recently, in relation to the CB2 receptor, it has been determined that different ligand agonists may specifically or predominantly activate specific receptor responses indicating a level of ligand functional selectivity for effects linked to the receptor.

Other cannabinoid receptors are found in almost every organ of the body including the skin, the digestive tract, and even in the reproductive organs. Cannabinoid receptors interact with cells in a lock (the cell receptor) and key (the cannabinoid) type of mechanism. The combination of the cell receptors and the cannabinoids comprise the endocannabinoid system, or ECS, which is an intricate network of cell receptor proteins that perform various functions in the body and is considered to be the greatest neurotransmitter system in the body. Bearing this in mind, it becomes important to realize and understand how cannabis can have an impact on numerous health issues including, but not limited to, Alzheimer's disease, memory loss, multiple sclerosis (MS) and other neurodegenerative diseases, and pain control and relief.

The major differences between the various cannabinoids are determined by the extent to which they are psychologically active (psychoactive). Three substantial classes of cannabinoids, including the cannabigerols (CBGs), cannabichromenes (CBCs), and cannabidiols (CBDs), are not known to have psychoactive effects. Δ-9-tetrahydrocannabinol (THC), cannabinol (CBN), and some other cannabinoids are known to be psychoactive to varying degrees. Non-psychoactive CBD is likely the most abundant cannabinoid, contributing up to 40% of cannabis resin in some strains (particularly those referred to as hemp strains); CBD has also been implicated in lessening the psychoactive effects of THC.

Of the over 80 known cannabinoid species, those most prevalent and most studied in cannabis cultivars are:
THC—Δ-9-tetrahydrocannabinol
CBD—cannabidiol
CBC—cannabichromene
CBN—cannabinol
CBG—cannabigerol
THCv—tetrahydrocannabivarin
CBDv—cannabidivarin
Δ-8-THC—Δ-8-tetrahydrocannabinol
THCA—Δ-9-tetrahydrocannabinolic acid
CBDA—cannabidiolic acid

A number of these 80-plus cannabinoids display a plurality of important medical effects. The subset of the aforementioned cannabinoids for which these medically beneficial effects are characterized and confirmed are presented here with their chemical formulae and structures.

THC: As used herein, THC refers to Δ-9-tetrahydrocannabinol, the chemical formula for which is C₂₁H₃₀O₂ and the structure of which is:

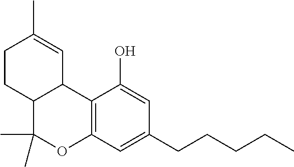 [SMILES: CCCCCc1cc(O)c2c(c1)OC(C)(C)C1CCC(C)=CC21]

THC is recognized as the primary psychoactive compound in cannabis and is the most common cannabinoid. Along with its psychoactive properties, THC may be medically used to alleviate several types of pain including the nerve-related pain of diabetic neuropathy and multiple sclerosis. Additionally, THC may be effective in alleviation of the symptoms of PTSD and reduction of nausea and vomiting, particularly that caused by chemotherapy. It has been shown to aid those with anorexia, as well as cancer and HIV associated wasting syndrome as it is an appetite stimulant. It improves breathing for asthmatics, acting as a potent bronchodilator, it relieves eye pressure in patients with glaucoma, improves insomnia, sleep apnea, and reduces nightmares. THC aids those with inflammatory bowel diseases such as Crohn's disease, ulcerative colitis and leaky gut, as well as other intestinal diseases by decreasing intestinal permeability and strengthening intestinal tight junctions. THC slows and prevents Alzheimer's disease and helps control seizures. THC reduces pain and tremors and improves sleep for those with Parkinson's disease. THC, CBD, CBG, and CBC together work synergistically as a powerful cancer tumor-fighting combination. This combination is more powerful than any single one of these working alone.

CBD: As used herein, CBD stands for cannabidiol, the chemical formula for which is C₂₁H₃₀O₂ and the structure of which is:

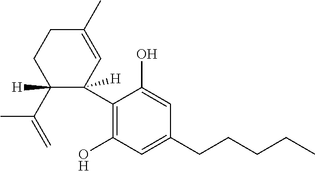

CBD, or cannabidiol, is a non-psychoactive member of the cannabinoids and is one of the most prevalent chemical compounds in the cannabis plant. Found predominantly in the resin glands of the female plant, this compound can stop muscle spasms and epileptic seizures, and can reduce idiopathic anxiety, a prevalent and significantly debilitating aspect of mental illness. It is used to treat nicotine addiction, osteoporosis, diabetes, cancer, obsessive-compulsive disorder, Lupus, Parkinson's disease, and motor disorders, and soothes neuropathic and chronic pain. It has anti-inflammatory, antioxidant, neuroprotectant, anxiolytic, antidepressant, analgesic, anti-tumor, and anti-psychotic effects. CBD is powerful all by itself, but it is even more powerful when combined with other cannabinoids such as THC.

CBC: As used herein, CBC stands for cannabichromene, the chemical formula for which is C₂₁H₃₀O₂ and the structure of which is:

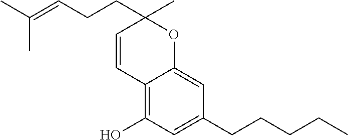 [SMILES: CCCCCc1cc(O)c2c(c1)OC(C)(CCC=C(C)C)C=C2]

Cannabichromene, or CBC, is the third most prevalent cannabinoid in the marijuana plant in general. In some strains CBC is more prevalent than CBD, and like CBD it is non-psychoactive.

CBC is anti-inflammatory and even more so when combined with THC. It has anti-tumor effects and shows promise in fighting breast cancer. When combined with CBD, THC, and CBG, the cancer fighting effects are intensified. It may be useful as an antidepressant and may be more powerful than the other cannabinoids in this capacity. CBC shows antiviral and mild antifungal activity. While CBC addresses several other health issues, including inflammation, cancer, depression, and fungal infections, it also increases the number of brain cells and therefore is useful in the treatment of several brain related disorders. CBC promotes neurogenesis in individuals at any age. This not only affects memory and learning, but can off-set certain dementias which occur when the brain stops growing new cells. It is likely that CBC can alleviate to some extent certain forms of depression and neuro-degenerative diseases via this particular mechanism of neurogenesis.

CBN: As used herein, CBN stands for cannabinol, the chemical formula for which is C₂₁H₂₆O₂ and the structure of which is:

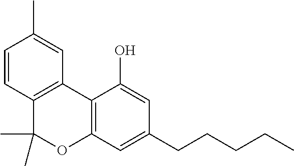 [SMILES: CCCCCc1cc(O)c2c(c1)OC(C)(C)c1ccc(C)cc1-2]

Cannabinol, or CBN, emerges when the dried cannabis flower becomes stale; over time, THC and CBD, which are generated by enzymatically-driven synthesis from CBG, break down through oxidative degeneration into CBN. CBN has antibiotic properties, including against methicillin-resistant Staphylococcus aureus (MRSA), and also has pain-relieving properties through the release of endorphins. It may delay the onset of, and relieve symptoms of, degenerative motor neural diseases such as amyotrophic lateral sclerosis (ALS) and MS. It works as an appetite stimulant and is more powerful than CBD and CBG in this regard. It has been found to have potent sedative characteristics, making it possibly the most potent single sedative of all the cannabinoids. When combined with THC, CBN has also been found to be effective at lowering the ocular pressure which produces blindness in glaucoma patients. CBN also promises to be useful in future for lowering blood pressure overall.

CBG: As used herein, CBG stands for cannabigerol, the chemical formula for which is C₂₁H₃₂O₂ and the structure of which is:

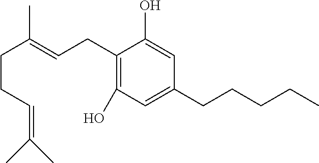 [SMILES: CCCCCc1cc(O)c(C/C=C(\C)CCC=C(C)C)c(O)c1]

CBG, or cannabigerol, is found in cannabis early in the growth cycle, making it somewhat difficult to find in large quantities (CBG is synthesized from smaller constituents, and serves as the feedstock for enzymatic synthesis of THC, CBD, and other cannabinoids, and so CBG is regarded as the source of all cannabinoids. It is non-psychoactive and can also be cultivated in hemp, in which it occurs in greater quantities. CBG has antibiotic properties stronger than CBN and comparable to CBD and is effective against various types of bacteria and fungi. It has therapeutic potential for skin conditions like psoriasis and eczema. CBG is reportedly a more potent pain reliever than THC, and functions as an antidepressant and mood-stabilizer by preventing the uptake of GABA and by increasing serotonin levels in the brain.

THCV: As used herein, THCV stands for tetrahydrocannabivarin, the chemical formula for which is C₁₉H₂₆O₂ and the structure of which is:

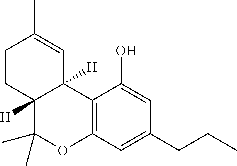 [SMILES: CCCc1cc(O)c2c(c1)OC(C)(C)[C@@H]1CCC(C)=C[C@@H]21]

THCV, or tetrahydrocannabivarin, is one of the several cannabinoids that works in synergy with THC, and mitigates some of the negative psychoactive impacts of THC. THCV's medical uses are antiepileptic, anticonvulsant, and anti-seizure; it is neuroprotective and mitigates some of the short-term memory and speech impairment that comes from THC; it promotes weight loss by suppressing the appetite and possibly decreasing body fat and boosting energy metabolism.

CBDV: As used herein, CBDV stands for cannabidivarin, the chemical formula for which is C₁₉H₂₆O₂ and the structure of which is:

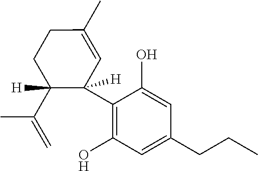

Cannabidivarin, or CBDV, is a slightly-degraded close relative of CBD. It is used as an anticonvulsant, an antiepileptic, and has antiemetic properties (as well as aiding those with gastrointestinal issues).

Δ-8-THC: As used herein, Δ-8-THC stands for Δ-8-tetrahydrocannabinol, the chemical formula for which is C₂₁H₃₀O₂ and the structure of which is:

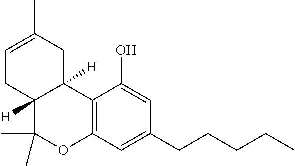 [SMILES: CCCCCc1cc(O)c2c(c1)OC(C)(C)[C@@H]1CC=C(C)C[C@@H]21]

Δ-8-tetrahydrocannabinol is different from Δ-9-tetrahydrocannabinol in that it is less psychoactive. It has both neuroprotective and anti-anxiety properties, as well as being anti-emetic, and may be a stronger appetite stimulant than Δ-9-THC, making it an important consideration for people undergoing chemotherapy.

THCA: As used herein, THCA stands for Δ-9-tetrahydrocannabinolic acid, the chemical formula for which is C₂₂H₃₀₀₄ and the structure of which is:

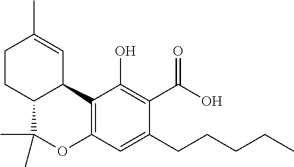 [SMILES: CCCCCc1cc2c(c(O)c1C(=O)O)[C@@H]1C=C(C)CC[C@H]1C(C)(C)O2]

Δ-9-tetrahydrocannabinolic acid, or THCA, is a non-psychoactive compound found in cannabis prior to decarboxylation to the psychoactive version, THC, by application of heat or drying or both. THCA levels are particularly high in the live or freshly harvested plant, but as the plant dries, THCA slowly converts to THC, a process expedited by smoking or vaping. Because THCA readily converts to the psychoactive THC upon heat application such as smoking or vaping, it cannot be inhaled or absorbed into the body by these particular means. THCA shows anti-inflammatory properties and may thus be used in treatment of arthritis and lupus. Its neuroprotective properties may make THCA a candidate for treatment of neurodegenerative diseases; its anti-emetic properties making it a possible treatment for nausea and appetite loss, and its anti-proliferative properties making it a candidate in treatment in certain cancers such as but not limited to prostate cancer.

CBDA: As used herein, CBDA stands for cannabidiolic acid, the chemical formula for which is C₂₂H₃₀O₄ and the structure of which is:

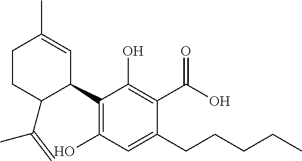 [SMILES: C=C(C)C1CCC(C)=C[C@H]1c1c(O)cc(CCCCC)c(C(=O)O)c1O]

Cannabidiolic acid, or CBDA, cannot administered by smoking or vaporizing because doing so decarboxylates it to CBD, similar to THCA conversion to THC. The therapeutic uses for CBDA include antibacterial, anti-emetic, anti-inflammatory, and cancer cell anti-proliferative.

Another family of active compounds present in, but not exclusive to, cannabis are the terpenes and decarboxylated terpenes, which are known as terpenoids. Decarboxylation occurs with the removal of the COOH functional group, and can be seen in drawings of the structures. These two terms are commonly used interchangeably, and although they are not chemically identical in structure or chemical formula as terpenoids are decarboxylated versions of some terpenes and exist in plants in this decarboxylated form, for the purposes of this invention both will be referred to as simply terpenes. Though cannabis contains up to 200 different terpenes and terpenoids, there are approximately 10 primary terpenes and 20 secondary terpenes that occur naturally in significant concentrations in the cannabis plant.

Terpenes are vital components of cannabis, and are important medicinally active compounds that are found in up to 1.5% of the total extraction. They are a large and diverse class of organic compounds, produced by a wide variety of plants giving them their flavor, aroma, and color. Terpenes are the building blocks of a plant's essential oils, and essential oils contain mixtures of the various terpenes found in the plants from which they were extracted.

The isoprene skeleton (C₅H₈) may be found in naturally occurring terpenes (also known as isoprenoids), but these terpene compounds do not arise from isoprene itself. Terpenes may be thought of as multiples of isoprene subunits, which is the cornerstone of the “isoprene rule” for terpenes.

The ten primary terpenes and twenty secondary terpenes that occur in significant concentrations are as follows:

The primary terpenes are: myrcene, α-pinene, ocimene, terpineol, β-caryophyllene, linalool, limonene, terpinolene, valencene, and geraniol.

The secondary terpenes are: phellandrene, carene, terpinene, fenchol, borneol, bisabolol, phytol, camphene, sabinene, camphor, isoborneol, menthol, cedrene, nerolidol, guaiol, isopulegol, geranyl acetate, cymene, eucalyptol, and pulegone.

These terpenes have non-psychoactive therapeutic effects and may be safely used to treat a variety of health conditions. They may also be combined with each other and with cannabinoids, yielding a whole new range of health effects. Some combinations of terpenes act in synergy with boosting effects, while others act as antagonists with effects that inhibit. Some terpenes increase the assimilation of THC, while others may affect the flow of dopamine and serotonin, two of the main regulators of mood and behavior.

Cannabinoid-terpenoid interactions have the potential to produce synergy with respect to the treatment of pain, inflammation, depression, anxiety, addiction, mood and behavior, epilepsy, cancer, fungal infections and bacterial infections, including MRSA.

The primary and secondary terpenes with some of their medical actions are as follows:

### Primary Terpenes:

Myrcene-Myrcene, specifically β-myrcene, is a monoterpene and the most common terpene produced by cannabis (some varieties contain up to 60% β-myrcene as a fraction of the total terpene content). A-myrcene is not found in nature and was first synthesized in 1965. The chemical formula for β-myrcene is C₁₀H₁₆ and the structure is:

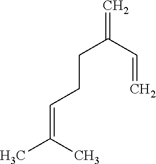

Myrcene is found in most varieties of cannabis as well as menthol, lemon grass, and hemp, and is widely used in the perfume industry. Its aroma has been described as musky, earthy, and herbal.

Myrcene has some very special medicinal properties, including lowering the resistance across the blood-brain barrier allowing itself and many other chemicals to cross the barrier more easily and quickly. Myrcene also increases cell membrane permeability, and in the case of cannabinoids like THC, β-myrcene allows the cannabinoid to take effect more quickly. More uniquely still, β-myrcene has been shown to increase the maximum saturation level of the CB1 receptor, allowing for a greater maximum psychoactive effect. Myrcene has anti-microbial and anti-septic properties, and acts as a natural anti-depressant, anti-carcinogen and anti-inflammatory agent. It is a potent analgesic and is anti-mutagenic. It blocks the action of cytochrome, aflatoxin B and other pro-mutagenic carcinogens. It acts as an inhibitor of gastric and duodenal ulcers. Its sedative and relaxing effects make it ideal for the treatment of insomnia and pain.

α-Pinene-Alpha-pinene is a monoterpene alkene isolated from pine needle oil as well as from cannabis. There are two structural isomers of pinene found in nature: α-pinene and β-pinene, with α-pinene being the most widely encountered terpenoid in nature. With an aroma and flavor of pine, this is partially where pine trees get their scent. The chemical formula is C₁₀H₁₆ and the structure is:

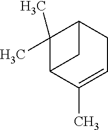

Pinene is one of the principal monoterpenes that is important physiologically in both plants and animals. It tends to react with other chemicals, forming a variety of other terpenes (like limonene), as well as other compounds.

Medicinally, α-pinene has an anti-tumor effect and has shown anti-cancer activity. Alpha-pinene is used as an anti-inflammatory, expectorant, bronchodilator, memory enhancer, as a local antiseptic, and it may decrease oil production in oily skin. It acts as a broad spectrum antibiotic and is highly effective against MRSA when combined with the cannabinoids CBD and CBN, all three working in synergy with each other. Alpha-pinene increases alertness and counteracts some of the negative effects of the cannabinoids THC, such as anxiety. It is also believed that the negative memory effects of THC may be lessened if mixed with α-pinene.

Ocimene—Ocimene is a group of isomeric monoterpenes found in a wide variety of fruits, spices, and plants. Alpha-ocimene and the two β-ocimenes, cis-β-ocimene and trans-β-ocimene, differ in the position of the isolated double bond: in the alpha isomer it is terminal. β-ocimene exists in two stereoisomeric forms, cis and trans, with respect to the central double bond. Ocimene is often found naturally as a mixture of its various forms. The chemical formula is C₁₀H₁₆ and the three structures are:

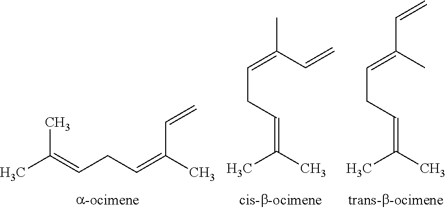

Ocimene is recognized by its sweet, fragrant, herbaceous, and woodsy aromas, which feature prominently in several perfumes as well as flavorings, and which help plants defend themselves in their natural environment. Ocimene occurs naturally in botanicals as diverse as cannabis, mint, parsley, pepper, basil, mangoes, orchids, kumquats, and allspice.

Ocimene's potential medical benefits include antiviral, antifungal, antiseptic, decongestant, and antibacterial benefits.

Terpineol-Found in cannabis as well as in over 150 other plants, terpineol exists as four isomers: α-terpineol, β-terpineol, γ-terpineol and terpinen-4-ol, are four closely related monoterpene alcohols. These are found mixed in plants and their essential oils, with α-terpineol comprising the majority of the mixture. The chemical formula is C₁₀H₁₈O and the four structures are:

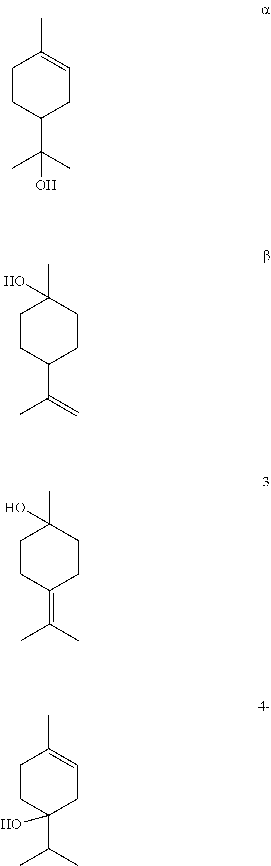

Terpineol has a floral aroma, resembling lilacs, clove, citrus, or apple blossoms, and other than cannabis it also occurs naturally in lilacs, pine trees, lime blossoms, and eucalyptus, as well as contributing to the distinctive, pine smoke-based aroma of lapsang souchong tea. From a flavor perspective, terpineol tastes like mint and anise. Terpineol is most frequently found in cannabis strains which also contain high levels of α-pinene. Due to α-pinene's strong aroma, terpineol may be difficult to detect by odor when the two occur simultaneously as the scent of α-pinene masks the more delicate floral scent of terpineol.

Terpineol, specifically α-terpineol, is known to have calming, relaxing effects and is a mild sedative. Terpineol inhibits skin acne, acts as an antibiotic, anti-inflammatory, antioxidant and has anti-malaria properties. Terpineol's most important property is its anti-cancer property′ it is able to kill tumors directly.

β-Caryophyllene—β-caryophyllene is a bicyclic sesquiterpene with the formula C₁₅H₂₄ and the structure:

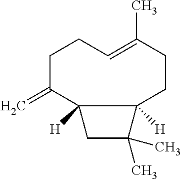

β-Caryophyllene (BCP) is a natural bicyclic sesquiterpene abundantly found in essential oils from various spices, fruits and medicinal as well as ornamental plants; β-caryophyllene is found in many plants such as various cannabis strains, copaiba, Thai basil, cloves, cinnamon leaves and black pepper, oregano, and other edible herbs; in minor quantities, it may be found in lavender as well as in many green, leafy vegetables. Its aroma has been described as peppery, woody spicy, and hoppy, as in hops used for brewing beer, to which cannabis is closely related. β-caryophyllene is the major terpene and active component of copaiba oil, which is known to exhibit strong antioxidant and anti-inflammatory action. In a study of the action of β-caryophyllene, the major constituent of copaiba oil, on the systemic inflammation, oxidative status, and liver cell metabolism associated with rheumatoid arthritis, it was found that β-caryophyllene reduced swelling of joints and lymph nodes as well as the number of circulating and articular leukocytes. Moreover, β-caryophyllene eliminated increases of protein carbonyl groups and myeloperoxidase activity in the liver and plasma of arthritic subjects and increased levels of reactive oxygen species and reduced glutathione in the arthritic liver. These beneficial actions were of the same type and strength as those of copaiba oil (Copaifera reticulata) and, therefore, β-caryophyllene is likely responsible for the anti-inflammatory and antioxidant actions of the oil.

β-caryophyllene is the only terpene known to interact with the endocannabinoid system, and does so at the CB2 receptor, which does not produce a high (that is, the CB-2 receptor is not implicated in cannabis psychoactivity). β-caryophyllene selectively binds to the CB2 receptor where it is a functional CB2 agonist, giving it an anxiolytic and anti-depressant effect and showing that β-caryophyllene may be useful in treating anxiety and depression. β-caryophyllene also has anti-oxidant, anti-inflammatory, anti-cancerous, and local anesthetic effects. Further, β-caryophyllene is unique for being both a terpene and a dietary cannabinoid, a food component which acts as a cannabinoid and binds to CB2 receptors.

Other phytocannabinoids in combination, especially cannabidiol (CBD) and β-caryophyllene, when delivered orally, appear to be promising candidates for the treatment of chronic pain due to their high safety and low adverse effects profiles. That is, the CB2 agonism of β-caryophyllene improves the therapeutic effectiveness of CBD synergistically. Therefore β-caryophyllene, through its CB2 receptor-dependent pathway, may be an excellent therapeutic agent to prevent nephrotoxicity (poisonous effect on the kidneys) caused by anti-cancer chemotherapy drugs such as cisplatin, in addition to its excellent anti-inflammatory and antioxidant effects.

β-caryophyllene also has antinociceptive properties, blocking sensory neuron detection of pain stimuli. This further suggests that copaiba oil or β-caryophyllene is useful in treating a number of medical issues such as arthritis and neuropathy pain. It is anti-inflammatory because of its ability to bind directly to the endocannabinoid receptor known as CB2. It is also protective of the cells lining the digestive tract, which offers promise for treating some ulcers, and is anti-fungal. β-caryophyllene holds promise for cancer treatment.

Copaiba oil is an oleoresin extracted from the trunk of the Copaifera tree and is widely used in popular medicine in that region for different purposes, such as anti-inflammatory, antitumoral and antimicrobial. Oil extracted from copaiba acts similarly as anti-inflammatory compounds (inhibiting histaminergic and serotoninergic pathways) and presents antinociceptive effect (possibly mediated by opioid receptors). Furthermore β-caryophyllene is a selective agonist to the peripheral cannabinoid receptor CB2, which is related to the treatment of pain and inflammation.

Linalool—Linalool is a terpene alcohol that occurs as two enantiomers d-linalool and 1-linalool, with the chemical formula C₁₀H₁₈O and the structures:

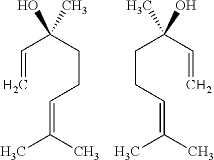

Linalool has a floral lavender aroma with a hint of spice. In addition to cannabis, linalool may be found in an array of flowers and spice plants such as lavender, bay laurel, sweet basil, mint, cinnamon, citrus and even some fungi. Linalool is a critical precursor in the formation of vitamin E.

Linalool may be used as an anti-inflammatory or as an immune booster, and may significantly reduce lung inflammation caused by cigarette smoke as well as reducing lung irritation potentially caused by inhaling cannabis smoke. Linalool helps to restore cognitive and emotional function partially via its anti-inflammatory effect, and may therefore be used to treat various forms of dementia, and particularly Alzheimer's disease. It helps with insomnia, and because it also lessens the anxiety brought on by pure THC, it helps in the treatment of anxiety and psychosis. Linalool has anesthetic effects and is calming, relaxing and mood lifting, and helps reduce headaches and migraines. Linalool may be useful to help treat liver cancer, and also helps to modulate motor movements, giving it anti-epileptic properties. It is an effective insecticide against fruit flies, fleas, and cockroaches, making it useful as an insect repellent and for use in and around the home and garden.

Limonene—Limonene is a monocyclic monoterpene and one of two major compounds formed from pinene. It exists as two enantiomers, d-limonene and 1-limonene, and has the chemical formula C₁₀H₁₆. The structures are:

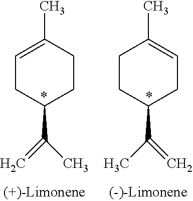

Limonene has a citrusy aroma and the more common d-isomer smells like oranges. While it is found in cannabis, it is also present in citrus fruit and especially lemons, juniper, and peppermint. It assists in the absorption of other terpenes through the skin and other body tissues. Limonene has anti-fungal, anti-bacterial, and anti-depressant effects; it promotes a general uplift in mood and attitude, and it helps promote weight-loss. It is a strong antioxidant and exerts anti-carcinogen properties as it may reduce the formation of some tumor growths and alleviate fat buildup in the liver induced by diet. Limonene is known to increase blood pressure which is useful for those with low blood pressure. It has very low toxicity and adverse effects are rarely associated with it.

Terpinolene—Terpinolene, also called δ-terpinene (δ-terpinene), is one of a class of isomeric monoterpenes, all of which have the chemical formula C₁₀H₁₆, and which differ from each other only in the position of the carbon-carbon double bonds. The α-terpinene, γ-terpinene, δ-terpinene (terpinolene) are all found in plant essential oils, whereas β-terpinene is synthetically prepared from sabinene. The chemical structures are:

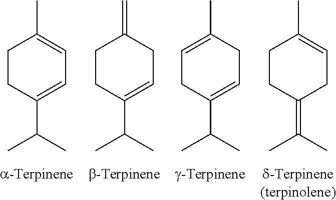

Terpinolene is characterized by a fresh, piney, floral, herbal, sometimes smoky or woody, and occasionally citrusy aroma and flavor. It is found in a variety of fragrant plants including cannabis, nutmeg, tea tree, conifers, citrus, apples, cumin, marjoram, sage, rosemary, Monterey cypress, and lilacs. It is used in soaps, perfumes, cosmetics, flavorings, and in the semiconductor industries.

Terpinolene is a central nervous system depressant used to induce drowsiness or sleep or to reduce psychological excitement. It has a sedative effect when inhaled, making it useful for insomnia and anxiety.

Terpinolene markedly reduces the expression of the AKT1 gene, which produces the protein AKT1 kinase, an enzyme that plays a vital role in various important signaling pathways and cellular processes. AKT1 kinase helps regulate cell growth and division (proliferation), differentiation, cell survival, and apoptosis (cell death) when cells become damaged or are no longer needed. The AKT1 gene belongs to the class of genes known as oncogenes. When mutated, oncogenes have the potential to cause normal cells to become cancerous. The activation of AKT is connected with many types of cancers as it increases cell proliferation and suppresses apoptosis. By suppressing the AKT1 gene expression, both rampant cell proliferation and lack of apoptosis are suppressed, making terpinolene a valuable anti-cancer agent.

Terpinolene, together with vitamins A and E, prevents the oxidation of “bad cholesterol” (low-density lipoprotein, or LDL) and is therefore helpful in the treatment of heart disease.

Terpinolene's potential medical benefits include: antioxidant, sedative, antibacterial, antifungal, insect repellent, anti-proliferative (anti-cancer) and non-genotoxic, making it very safe and very healing.

Valencene—Valencene is a bicyclic sesquiterpene with chemical formula C₁₅H₂₄ and is found in Valencia oranges as well as cannabis. The chemical structure is:

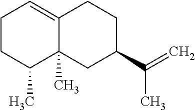

It has a sweet, fresh, citrusy, woody, aroma and flavor and is used in both the flavor and perfume industries.

Valencene is toxic to ticks and mosquitoes at lesser concentrations than DEET and doesn't have the toxicity of DEET. Valencene is an effective insect repellent for ticks, mosquitos, and other insects. It is also anti-inflammatory, and may lower the levels of inflammatory markers in macrophages.

Geraniol—Geraniol is an acyclic monoterpene alcohol whose formula is C₁₀H₁₈O and which boils at about 447° F. and frequently occurs in strains that also produce linalool. Not only from cannabis, geraniol is also found in rose, geranium, lime, lemon, lemongrass, nutmeg, bergamot, carrot, coriander, lavender, blueberry, blackberry, and tobacco. Geraniol emits a rose-like scent that makes it a popular perfume additive. The chemical formula is:

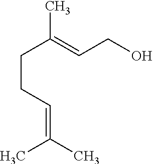

Geraniol is an effective mosquito repellent, an antioxidant, and shows a potential protective effect against neuropathy. It is anti-cancer and inhibits the growth and biosynthesis of colon cancer cells, and when combined with farnesol and perill alcohol, suppress pancreatic tumor growth making it especially useful for cancer of the pancreas which currently is extremely difficult to cure.

### Secondary Terpenes:

Phellandrene-Phellandrene refers to a pair of cyclic monoterpenes that have a similar molecular structure and similar chemical properties, α-phellandrene and β-phellandrene, which are double-bond isomers of each other. In α-phellandrene, both double bonds are endocyclic (within the ring structure) and in β-phellandrene, one of them is exocyclic (external to the ring structure). Phellandrene has the chemical formula C₁₀H₁₆ and is described as pleasant, fresh, citrusy, minty and peppery-woody. The chemical structures are:

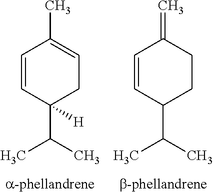

Phellandrenes are used in the perfume and the flavoring industries because of their pleasing aromas and because they are absorbed through the skin. α-phellandrene may form dangerous, explosive peroxides on contact with air at elevated temperatures. β-phellandrene is non-hazardous, and both phellandrenes may be found in cannabis as well as in spices such as allspice, cinnamon, garlic, dill, pepper, parsley, and in the essential oils of angelica, eucalyptus, lavandula, mentha, fennel, ginger, and Pinus species.

Insoluble in water but miscible with ether, phellandrene is one of the easiest terpenes to identify in the lab. When a solution of phellandrene in a solvent (or an oil containing phellandrene) is treated with a concentrated solution of sodium nitrate and then with a few drops of glacial acetic acid, very large crystals of phellandrene nitrate speedily form.

Phellandrene has special medicinal values and has been used in traditional Chinese medicine to treat digestive disorders. It is one of the main compounds in turmeric leaf oil, which is used to prevent and treat systemic fungal infections. Phellandrene possesses antidepressant properties and is also used as an insecticide.

Carene—Δ-3-Carene is a bicyclic monoterpene with a sweet, pungent odor. It is found naturally in cannabis and in many healthy, beneficial essential oils, including cypress oil, juniper berry oil and fir needle essential oils, and is a main constituent of pine and cedar resin. It is also present in bell pepper, basil oil, grapefruit and orange juices, citrus peel oils from fruits like lemons, limes, mandarins, tangerines, oranges, kumquats, and it is a major component of turpentine, comprising as high as 42% depending on the source. The chemical formula is C₁₀H₁₆ and the chemical structure is:

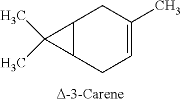

Δ-3-Carene is used as a flavoring in many products.

It is nontoxic but may cause irritation when inhaled. It is possible that high concentrations of δ-3-carene in some strains may be partly responsible for symptoms of coughing, itchy throat, and eye afflictions when smoking cannabis.

Δ-3-carene is an effective anti-inflammatory. In higher than natural concentrations, δ-3-carene may be a central nervous system depressant and a skin irritant. It is often used to dry out excess body fluids, such as tears, runny noses, sweat, and menstrual flows.

Terpinene—Terpinenes are a group of isomeric terpenes with the chemical formula C₁₀H₁₆ and this group is composed of three natural isomeric terpenes and one synthetic one that differ from each other in the positions of the carbon to carbon double bond. Δ-terpinene, δ-terpinene (terpinolene), and γ-terpinene are naturally occurring, whereas β-terpinene is not found in nature but may be synthetically produced from sabinene. Δ-terpinene is also called erpinolene.

### The Chemical Structures are:

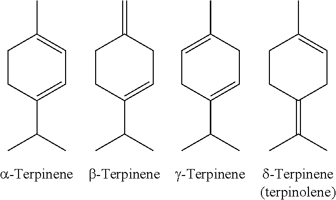

Terpinene is a major component of essential oils made from citrus fruits, and has a lemon odor. Δ-terpinene is widely used in the flavor, perfume, cosmetics, soap, pharmaceutical industries, as well as in food and confectionary.

Terpinene is considered to be a well-tolerated additive in the pharmaceutical industry, and it has very strong antioxidant properties.

Fenchol—Fenchol, also called 1,3,3-trimethyl-2-norbornanol, is a terpene and an isomer of borneol with the chemical formula C₁₀H₁₈O and the chemical formula is:

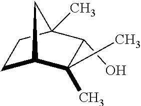

This particular terpene is an enantiomer, d-fenchol or (1R)-endo-(+)-fenchol, but it has no mirror image found in nature, thus it is enantiopure.

Found in cannabis, it also occurs naturally in basil, fennel, nutmeg, pine, rosemary oil, lime oil, beer and more. It has a bitter, lime flavor and is used extensively in perfumes, flavorings, soaps, detergents, and personal care products. It is known to exhibit antibacterial properties.

Borneol—Borneol, a terpene alcohol, has the chemical formula C₁₀H₁₈O and exists naturally as two enantiomers, 1-borneol and d-borneol, both of which are found in nature. It is easily oxidized to camphor, has an aroma of camphor, mint, and earth, and is a component of many natural essential oils. It is found in cannabis resin and herbs like thyme, rosemary, and cinnamon. The chemical structure is:

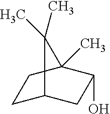

Borneol is used in the perfume industry, as well as in dietary and herbal supplements in the USA.

Borneol is used as a calming sedative, it is used to fight fatigue, stress, to relax, and to recover from illness. Borneol is used as an anti-inflammatory, an anti-nociceptive/analgesic, a skin tonic, a local anesthetic, as an anti-insomnia, anti-septic, a digestive aid, a sedative and an antispasmodic. It is used to improve circulation, to reduce pain and swelling, as a bronchodilator, a cough suppressant, and an insect repellant.

Bisabolol—Also called levomenol, α-bisabolol is a natural monocyclic unsaturated sesquiterpene alcohol with the chemical formula C₁₅H₂₆O and a chemical structure of:

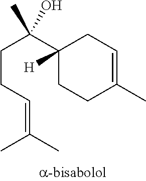 [SMILES: CC(C)=CCC[C@](C)(O)[C@@H]1CC=C(C)CC1]

A-bisabolol is found in cannabis, the Brazilian shrub candeia, and German chamomile. It has a floral aroma.

A-bisabolol, which is nontoxic and nonirritating to the skin, possesses anti-inflammatory and wound healing properties, as well as antimycotic and antibacterial effects, and may be used as a deodorizer. It is a potent inhibitor of fungi, Candida albicans, and gram-positive bacteria. It shows promise in the treatment of certain cancers as it induces apoptosis in leukemia.

Phytol—Phytol is a natural linear diterpene alcohol with the chemical formula C₂₀H₄₀O that may be used as a precursor to prepare synthetic forms of vitamin E and vitamin K1. Found in cannabis and green tea, phytol results from the degradation of chlorophyll and is an oily liquid that is nearly insoluble in water, but soluble in most organic solvents. The chemical structure is:

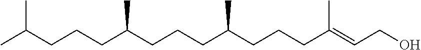 [SMILES: C/C(=C\CO)CCC[C@H](C)CCC[C@H](C)CCCC(C)C]

Phytol inhibits the enzyme that degrades the neurotransmitter GABA (γ-aminobutyric acid), which may partially account for its relaxing effect. In the body, phytol is essential in activating enzymes that have a positive effect on the production of insulin. It is beneficial in regulating blood glucose, for reducing blood pressure and for reducing cholesterol levels in blood.

Camphene—Camphene is a bicyclic monoterpene with the chemical formula C₁₀H₁₆ and the chemical structure:

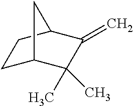

Camphene readily volatilizes at room temperature and has a pungent odor similar to camphor. It is a minor component of many essential oils such as turpentine, cypress, neroli, valerian camphor, citronella and ginger. It is used as a flavoring for food, and in the perfume industry. It is produced industrially by catalytic isomerization of the more common α-pinene.

Camphene is found in essential oils extracted from cannabis and certain trees, and it may play a critical role in cardiovascular health. Camphene possesses antioxidant, anti-inflammatory, and antibiotic characteristics, and shows promise for pain relief.

Camphene may reduce plasma cholesterol and triglycerides. Given the importance this plays in heart disease, camphene might be used as an alternative to pharmaceutical drugs which cause intestinal problems, liver damage, and muscle inflammation.

Sabinene-Sabinene is a bicyclic monoterpene with the chemical formula C₁₀H₁₆, and exists as d and 1 enantiomers. The chemical structures are:

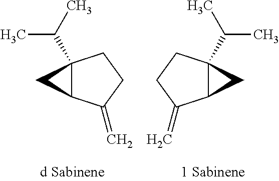

It has an aroma of spice, pine, and orange, and is found in many plants including cannabis, Norway spruce, black pepper, basil, and Myristica fragrans—the world's main source of nutmeg. It is used in the perfume industry and in the food industry as a flavoring.

Sabinene has antioxidant and anti-inflammatory properties, and benefits liver function, digestion, relieves arthritis, and may soothe skin conditions.

Camphor—Camphor is a waxy, flammable, white crystalline solid with the chemical formula C₁₀H₁₆O. Camphor occurs naturally as d-camphor, the 1-enantiomer being synthetically produced.

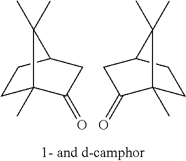

It is commonly found in cannabis, rosemary leaves, camphor basil, and in Cinnamomum camphora, which goes by several common names including camphor tree, camphorwood, and camphor laurel. Camphor is also found in kapur trees, and a few other related trees in the laurel family, notably Ocotea usambarensis.

The ancient Egyptians used camphor as one of the ingredients used for mummification. It has been used as an ingredient in sweet and savory foods in medieval Europe and Arabia. Camphor is readily absorbed through the skin, and when applied topically produces a cooling sensation similar to that of menthol. It acts as a slight local anesthetic, relieves pain, itching and swelling, and has antimicrobial properties. It is used as a cough suppressant, a decongestant, an insect repellant notably for cockroaches and fleas, and is used to make mothballs. Camphor has been used to treat sprains, swellings, inflammation, and fevers. In very small quantities taken internally, it is used to treat minor heart symptoms and fatigue. Camphor increases heart rate, is a skin vasodilator, and reduces appetite.

Isoborneol—Isoborneol is a bicyclic terpene alcohol with the chemical formula C₁₀H₁₈O and the chemical structure:

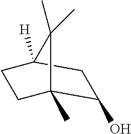

Isoborneol is a waxy solid with an odor similar to that of camphor and is found in cannabis and mugwort. Isoborneol exhibits antiviral properties and is a potent inhibitor of herpes simplex virus type 1. Besides being antiviral, it also has antioxidant, anti-inflammatory, and antimicrobial properties.

Menthol-Menthol is a terpene alcohol with the chemical formula C₁₀H₂₀O and the chemical structure:

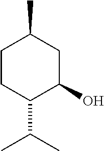

Menthol is found in cannabis and in members of the mint family such as corn mint and peppermint (Menthae piperitae aetheroleum). Menthol is a white or colorless crystalline solid at room temperature. It is used in candies, cigarettes, cosmetics, personal care products, and medicines.

Menthol produces a cooling sensation on the skin and soft tissues of the mouth by activating the TRPM8 receptor protein that senses the change in temperature in cold-sensing nerves. However, menthol gives a cool sensation without any actual fall in temperature in that area. This lowers inflammation in the area, causing the nearby blood vessels to dilate, and increases blood flow to the area which delivers fresh nutrients to repair the area and removes any toxic wastes generated. This process speeds healing. Menthol may also bind to another receptor called kappa opioid receptor that may also produce a numbing effect.

Menthol exhibits analgesic properties and is used topically to treat inflammatory pain caused by conditions such as arthritis, bursitis, tendonitis, muscle strains or sprains, backache, bone pain, bruising, and cramping.

Menthol cigarettes have a lower cancer risk and cause far less cigarette related cancers than their non-mentholated counterparts, making menthol an important and possibly mitigating component of inhaled cannabis.

Menthol is also a powerful penetration enhancer (PE) for transdermal drugs, often increasing drug uptake by a factor of 10 or more.

Cedrene—Cedrene is a sesquiterpene with the chemical formula C₁₅H₂₄ and exists in two isomeric forms, α-cedrene and-β-cedrene, which differ in the position of one double bond.

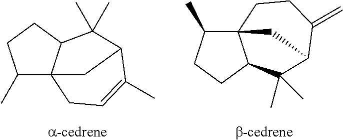

Cedrene is a light yellowish transparent oil with the aroma of cedar wood and is found in cannabis, fenugreek, and in the essential oil of cedar.

Cedrene possesses antiseptic, antimicrobial, antifungal, and anticancer properties, particularly against T-cell lymphoma, which may occur in the blood as leukemia or in lymph nodes (lymphoma), skin, or other areas of the body.

Nerolidol—Also known as peruviol, nerolidol is a naturally occurring sesquiterpene alcohol present in various plants with a floral odor, and has the chemical formula C₁₅H₂₆O. It exists in two isomeric forms, cis and trans, which differ in their geometry about the central double bond. The chemical structures are:

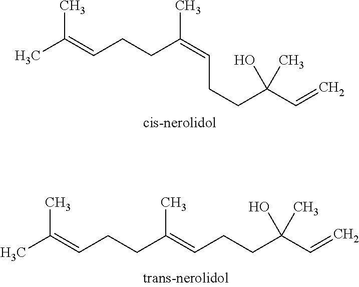

Nerolidol has a floral, citrus, woody, fresh bark aroma, and may be found in Cannabis sativa, neroli, niaouli, ginger, jasmine, lavender, tea tree, citronella, lemon grass, and Brassavola nodosa, a Mexican orchid.

Nerolidol is widely used in perfumes as both a base note fragrance component and as a fixative; it is also used in cosmetics, personal care products, detergents and cleaning products, and as a food flavoring agent.

It has anti-fungal, anti-leishmaniasis (an infection caused by protozoan Leishmania parasites, which are spread by the bite of phlebotomine sand flies) and anti-malarial properties. It also produces a sedative effect. It may enhance skin penetration for the transdermal delivery of therapeutic drugs.

Guaiol—Guaiol, also called champacol, is a sesquiterpenoid alcohol found in several plants, including Cannabis indica, guaiacum and cypress pine. It is a crystalline solid at room temperature with the chemical formula C₁₅H₂₆O and the structure:

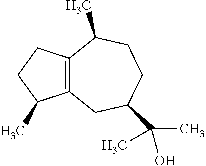 [SMILES: C[C@H]1CC[C@@H](C(C)(C)O)CC2=C1CC[C@@H]2C]

Guaiol has a woody, rosy, floral aroma. Cannabis strains known to contain guaiol include Liberty Haze, Blue Kush, Chocolope, and Medical Mass.

Guaiol has been used for centuries as a treatment for diverse ailments ranging from coughs to constipation to arthritis and syphilis. It is also an effective insect repellent and insecticide. Guaiol's potential medical properties include: Antimicrobial, Anti-inflammatory, laxative, diuretic, and insect repellant.

Isopulegol—Isopulegol is a monoterpene alcohol found in cannabis, corn mint, European pennyroyal, lemongrass and geranium, and possesses a minty aroma. It has the chemical formula C₁₀H₁₈O and the structure:

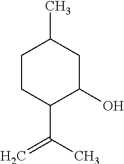

Isopulegol is used as a flavoring agent in food, in cosmetics, and in perfumes, personal care products, and cleaners. It is a chemical precursor to menthol and shows many promising routes for therapeutic use. Isopulegol possesses gastroprotective, anti-convulsive, anti-inflammatory, antioxidant, and stress-reducing effects, and it reduces the severity of seizures and anxiety in animal models.

Geranyl Acetate—Geranyl acetate has several other names including geraniol acetate, and is a monoterpene ester with a sweet, strong, floral rose and fruity aroma. It is a colorless liquid at room temperature and has the chemical formula C₁₂H₂₀O₂ with the structure:

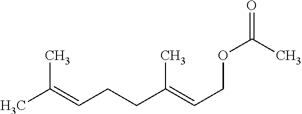

It is used in the fragrance and flavor industries, and is found in products such as soaps, detergents, personal care products, fabric softeners, and as a middle note in perfumes.

Geranyl acetate is found in a variety of natural essential oils, such as cannabis, citronella, palmarosa, geranium, coriander, neroli, lemongrass, petitgrain, carrot, sassafras, rose, and many others. It exhibits strong antimicrobial, antifungal, and anti-inflammatory effects.

Cymene—Also called p-cymene, para-cymene, methyl-isopropyl-benzene, and 1-isopropyl-4-methylbenzene among others, this aromatic, para substituted benzene ring is an alkylbenzene monoterpene with the formula C₁₀H₁₄ and the structure:

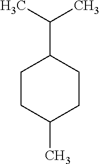

The other two isomers of methyl-isopropyl-benzene are o-cymene (ortho-cymene) and m-cymene (meta cymene), however only p-cymene is a naturally occurring compound. It has a citrusy-woody-spicy odor with herbal hints, and is found in cumin, thyme, anise, coriander, mace, oregano, eucalyptus and in angelica root and angelica seed oil, bay leaf oil, basil oil, carrot seed oil, clove bud oil, clary sage oil, and grape fruit oil. It is used in flavoring beverages, cakes and confectionery, as well as in the fragrance, paint, and furniture industries.

P-cymene has documented anti-inflammatory effects, it shows potential protective effects against acute lung injury, and is effective against pathogenic bacteria, especially Escherichia coli. When combined with carvacrol it is also antibacterial and possibly even more so. P-carvacrol, thymol and p-cymene work synergistically together and have anti-fungal properties; p-cymene by itself showed strong antifungal activity against numerous candida species. P-cymene also shows anti-inflammatory, antinociceptive and analgesic properties.

A useful derivative of cymene is 2,5-Dimethoxy-p-cymene, or thymohydroquinone dimethyl ether. 2,5-Dimethoxy-p-cymene is a phytochemical found in the essential oils of plants within the family Asteraceae, such as Arnica montana. These essential oils, which contain the compound as a major component of the oil, have antifungal, antibacterial, and insecticidal properties. Furthermore, 2,5-Dimethoxy-p-cymene appears to act synergistically with conventional chemotherapy and radiotherapy, and some clinical studies in humans have been initiated.

Eucalyptol—Eucalyptol has many other names, including 1,8-cineol (or cineole), cajeputol; 1,8-epoxy-p-menthane, and eucalyptole. Eucalyptol is a cyclic monoterpenoid ether and it is the main component of eucalyptus essential oil having the chemical formula C₁₀H₁₈O and the chemical structure:

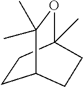

Eucalyptol has a minty, earthy, spicy aroma and is found in several plants including Cannabis sativa, camphor laurel, bay leaves, tea tree, mugwort, sweet basil, wormwood, rosemary, common sage, and other aromatic plants. Eucalyptol is used in flavorings in baked goods, confectionery, meat products, beverages, and mouth wash; in fragrances, cigarettes and cosmetics.

Eucalyptol has many medicinal uses. It relieves pain, suppresses coughs, and improves concentration and inner balance. Plants containing eucalyptol enhance meditation and concentration. Eucalyptol has potent antifungal effects and is used as an insecticide and insect repellent. Eucalyptol inhibits cytokine production in lymphocytes and monocytes, giving it an anti-inflammatory effect, and it reduces inflammation and pain when applied topically. It is able to kill in vitro leukemia cells of two cultured leukemia cell lines. Eucalyptol is effective for controlling asthma and reduces airway mucus hypersecretion by its anti-inflammatory cytokine inhibition, and it is an effective treatment for rhinosinusitis. Moreover, eucalyptol has been found to be a powerful penetration enhancer (PE) for transdermal drug delivery, often increasing drug intake by a factor of more than 10 via the transdermal route.

Pulegone—Pulegone, a monocyclic monoterpenoid, is a secondary terpene component of cannabis. It exists naturally in two enantiomeric forms, d-pulegone and 1-pulegone, with d-pulegone being the most abundant. The chemical formula is C₁₀H₁₆O and the structure is:

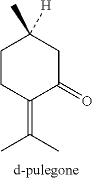

It has an aroma of peppermint and camphor, and it is found in several plants besides cannabis, such as catnip, peppermint, spearmint, pennyroyal, and rosemary. It is used for flavoring foods, drinks, and dental products, as a spice, it is used as fragrance components in detergents and cosmetics, it is used in herbal medicines, perfumery, and aromatherapy.

Pulegone is an emmenagogue, a mucolytic, and is good for congestion of the respiratory system. Pulegone may have significant sedative and fever-reducing properties. It may also alleviate the side effects of short-term memory loss sometimes associated with higher levels of THC. Pulegone is a powerful insecticide.

Traditionally, plants containing pulegone, such as pennyroyal, have been used as herbal teas for non-ulcer dyspepsia, primary dysmenorrhoea, secondary amenorrhoea and oligomenorrhoea, as an abortifacient, and as a diaphoretic. Pennyroyal essential oil has been used for the same conditions. Pulegone is a hepatotoxic (liver poison) and nephrotoxic (kidney poison) constituent of the folklore abortifacient pennyroyal oil.

Today, Mentha piperita (peppermint) and Mentha pulegium (pennyroyal) are used for colds, headache, migraine, as a diuretic, antispasmodic, anticonvulsive, anti-emetic, heart stimulant, sedative, and to treat the symptoms of inflammatory bowel syndrome. Rosemary inhibits acetylcholinesterase in the brain yielding more acetylcholine and allowing nerve cells to communicate more effectively with one another, giving promise for treatment of memory issues and dementias.

One other terpene found in cannabis that bears mentioning is humulene.

Humulene—Humulene is a monocyclic sesquiterpene containing an 11-membered ring and is also known as α-humulene and α-caryophyllene (an isomer of β-caryophyllene). Humulene is often found in combination with it's isomer, β-caryophyllene, it has the chemical formula C₁₅H₂₄ and the structure is:

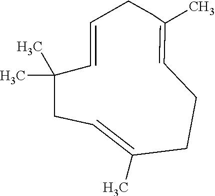 [SMILES: C/C1=C\CC/C(C)=C/CC(C)(C)/C=C/CC1]

Humulene is found in Cannabis sativa strains, hops and Vietnamese coriander, pine trees, orange trees, marsh elders, tobacco, sage, ginseng, ginger, and sunflowers, among other plants. Humulene is what gives beer its distinct “hoppy” aroma, and also contributes to the same hoppy aroma in cannabis.

Humulene is anti-tumor, anti-bacterial, is a strong anti-inflammatory, and is anorectic (suppresses appetite). It is often blended with β-caryophyllene and used as a potent remedy for inflammation. Humulene aids in weight loss by acting as an appetite suppressant.

In the body, terpenes act on receptors and neurotransmitters. They readily combine with, or dissolve in, lipids or fats. Terpenes may act as serotonin uptake inhibitors, they may enhance norepinephrine activity, they may increase dopamine activity, and they may augment synaptic γ-aminobutyric acid (GABA) levels by inhibiting re-uptake. These actions are similar to many of the commonly prescribed anti-depressant drugs used today.

The differences in the amounts and types of both cannabinoids and terpenes, along with the other lesser compounds within the cannabis varieties, imbue the various cannabis extracts with medicinal significance. Adding or increasing one or more of these compounds can alter the effects of cannabis extract, as certain compounds work in synergy to augment desirable effects while other compounds act as antagonists to inhibit undesirable effects.

Cannabis has been used to treat pain since at least the third millennium BC. In recent decades, and especially since changes in state laws allowing medical use of cannabis, there has developed a substantial body of anecdotal evidence that cannabis, particularly smoked cannabis plant matter, provides substantial relief from chronic pain caused by numerous chronic health conditions such as cancer, multiple sclerosis, arthritis, nerve damage, back pain, fibromyalgia, and similar conditions. In fact, smoked cannabis plant matter appears to be a preferred method of pain treatment by those with chronic medical conditions, even though other forms of administration of cannabis are available for medical use. This preference is likely due to a variety of factors. First, smoking cannabis allows the user to easily control the dosage, as the effects are felt quickly, and additional amounts can be smoked if the initial effects are insufficient. Second, the concentrations of active cannabis compounds in the bloodstream are high compared to oral administration, and more powerful, as the active cannabis compounds go directly into the bloodstream instead of being processed into other compounds by the liver. Third, the combined effect of the dozens of cannabis compounds in cannabis plant matter may provide a more pleasant subjective effect, or other benefits such as reduced inflammation, beyond that of a purified cannabinoid medical product. In an aspect of the embodiment, cannabis compounds can be selected in formulations designed to specifically to address each of these issues, while avoiding the detriments of smoked cannabis plant matter, such as respiratory irritation and the increased potential for lung disease. Further arguing in favor of specifically-formulated cannabis-based products is the fact that certain modern North American and European strains of cannabis display relatively high concentrations of THC (a potent psychoactive compound), but relatively little CBD or other phytocannabinoid content.

A substantial body of medical literature supports the anecdotal evidence that cannabis can be used to treat chronic pain. The endocannabinoid system is active in the control of pain. Compounds found in cannabis act on the endocannabinoid system, and certain of these compounds have a powerful analgesic effect. For example, THC is believed to be ten times more powerful than morphine in mediating pain in wide dynamic range neurons in the ventroposterolateral nucleus of the thalamus. Cannabidiol (CBD) is also believed to have strong analgesic effects, due to its function as an endocannabinoid modulator, likely through its ability to promote signaling of the adenosine receptor A2A by inhibiting the adenosine transporter. Cannabigerol (CBG), a “minor cannabinoid” found in small quantities in cannabis, is believed to have even greater analgesic activity than THC.

Chronic pain conditions are often resistant to standard treatments, including treatment with opioid medications such as morphine. There is some medical evidence that suggests that cannabinoids are complementary to treatment with opioids, offering additional pain reduction on top of that provided by the opioids. Combining cannabis treatments for chronic pain with opioid treatments for chronic pain may have the benefit of reducing patient pain levels, reducing reliance on (and addiction to) opioids, or both.

As mentioned above, a combination of hormone supplementation targeting increased HGH serum levels in conjunction with analgesic therapies and antibiotic/antifungal therapies, delivered transdermally, is desirable. Many uses of HGH supplementation—improved vitality in the elderly, improved performance amongst athletes, wasting disease such as amyotrophic lateral sclerosis (ALS), and myalgic encephalomyelitis/chronic fatigue syndrome (ME/CFS)—are also associated with the need for management of chronic pain or antimicrobial indications. For both topical and systemic purposes, the ability to transdermally deliver both hormone supplementation and treatment of local, topical, or chronic pain is beneficial and indeed preferable over hormone supplementation via injection or oral administration, as long as the skin barrier can be overcome in an appropriate way.

The inventor has conceived, and reduced to practice, a composition and methods of treatment using transdermal supplementation that overcome the limitations of transdermal delivery by various synergistic combinations of penetration enhancers, homeopathic formulations based on large biomolecules, and transdermal administration of hormone precursors in lieu of the actual large biomolecule itself. In some aspects, further methods of treatment of chronic or acute, systemic or local pain are also provided. In further aspects, antimicrobial treatments such as antibiotic, antiviral, and/or antifungal are provided by the addition of antimicrobial phytochemicals to a transdermal gel formulation.

In a preferred aspect, a transdermal gel formulation is provided that comprises a homeopathic formulation of somatropin in conjunction with the HGH precursor L-DOPA, which is more readily amenable to transdermal administration. In an aspect, the homeopathic formulation of somatropin is a 30D formulation. In some aspects, the homeopathic formulation of somatropin may be in the range of 24D-36D, or in a range of 18D-42D. In some aspects, L-DOPA is provided via velvet bean extract. In some aspects, velvet bean extract comprises less than 1% by weight of the transdermal gel formulation. In other aspects, velvet bean extract comprises between 0.1% and 0.9% by weight if the transdermal gel formulation.

In a preferred aspect, the transdermal gel formulation comprises one or more synergistic compounds that target skin health and improve absorption of the homeopathic formulation. Specifically, a synergistic mixtures of tocopherols and aloe vera gel may be used to deliver the homeopathic mixture while providing therapeutic benefits for the skin to which it is applied. In an aspect, alpha-tocopherol may be provided in an amount between 400-500 IU, combined with a balance of aloe vera gel. In this mixture, the antioxidant and skin protective benefits of the aloe vera gel as a mixture substrate are enhanced by the presence of alpha-tocopherol such that the resulting mixture's beneficial effects are greater than those of either ingredient individually. Additional tocopherols may be utilized as additives for specific purposes targeting certain effects.

In a preferred aspect, the transdermal gel formulation may further comprise therapeutic doses of milk thistle extract and carnosine, forming a synergistic mixture with the aloe vera gel and vitamin E tocopherols and tocotrienols present.

In a preferred aspect, the transdermal gel formulation comprises a gel-based delivery mixture to improve the absorption and bioavailability of the homeopathic formulation. Specifically, a gel comprising aloe vera gel with the addition of hyaluronic acid in the form of a cross-linked sodium hyaluronate polymer is provided. The aloe vera gel provides a water-based substrate in which the sodium hyaluronate polymer is gelled, while providing skin health benefits to the area to which the gel is applied. The sodium hyaluronate forms a cross-linked polymer that acts as an ideal delivery mechanism for the homeopathic formulation, enhancing absorption across cell membranes through endocytosis to afford a level of bioavailability not possible in non-gelled formulations.

In a preferred aspect, the transdermal gel formulation comprises one or more synergistic penetration enhancers comprising natural phytochemicals. Specifically, in one aspect eucalyptol is provided as a penetration enhancer. In an aspect, eucalyptol is provided in the form of eucalyptus in an amount of about between 0.5% and 1.5% by weight of the transdermal gel formulation. In some aspects, more or less eucalyptus oil may be used, such as between 0.1% and 0.5% or between 1.5% and 2%; in other aspects between 0.1% and 3% by weight of eucalyptus oil is included in the transdermal gel formulation. In another preferred aspect, menthol is used as a natural penetration enhancer, such as by incorporation of about 3% by weight of peppermint oil in the transdermal gel formulation. In some aspects, between 2.5% and 3.5%, or between 2% and 4%, or between 1% and 4%, or less than 5%, by weight, of peppermint oil may be used in the transdermal gel formulation. Moreover, it has been found that the penetration enhancement effects of eucalyptol and menthol are synergistic, such that for any given amount of each the total penetration enhancement is greater than the sum of the penetration enhancements of each used individually. This synergy allows for different mixtures to achieve similar penetration enhancement effects. For example, if it is desired to reduce the odor of mint provided by peppermint oil, or to reduce the known cooling effect of peppermint oil, or to reduce any skin irritation resulting from the peppermint oil on sensitive skin, the combination of both peppermint oil (for menthol) and eucalyptus oil (for eucalyptol) allows for relatively more eucalyptol and less menthol to achieve the desired effects without losing the penetration enhancement effects of either phytochemical. The combination of eucalyptol and menthol may improve transdermal penetration of the phytochemicals in the transdermal gel formulation by a factor of 10 to 20 times.

It should be noted that each phytochemical exhibits strongly synergistic effects vis-à-vis the others in the transdermal gel formulation. For example, menthol not only is a potent penetration enhancer but also provides substantial antimicrobial support against viruses, bacteria, and fungi, and it is strongly anti-inflammatory. Thus menthol is very well-suited for gel applications involving healing of skin, wounds, or other tissues via topical gel application. Furthermore, menthol exhibits analgesic properties and the transdermal gel formulation may be used topically to treat inflammatory pain caused by conditions such as arthritis, bursitis, tendonitis, muscle strains or sprains, backache, bone pain, bruising, and cramping.

Similarly, in addition to being a potent penetration enhancer that operates synergistically with menthol, eucalyptol has many medicinal uses. It relieves pain, suppresses coughs, and improves concentration and inner balance. Plants containing eucalyptol enhance meditation and concentration. Eucalyptol has potent antifungal effects and is used as an insecticide and insect repellent. Eucalyptol inhibits cytokine production in lymphocytes and monocytes, giving it an anti-inflammatory effect, and it reduces inflammation and pain when applied topically. It is able to kill in vitro leukemia cells of two cultured leukemia cell lines. Eucalyptol is effective for controlling asthma and reduces airway mucus hypersecretion by its anti-inflammatory cytokine inhibition, and it is an effective treatment for rhinosinusitis.

In a preferred aspect, CBD is included in the transdermal gel formulation for its strong medicinal effects that are complementary to, and in several ways synergistic with, the effects of the hormone supplement therapeutics (homeopathic somatropin and/or L-DOPA) and the eucalyptol and menthol. In particular, it is well-established that CBD soothes neuropathic and chronic pain and has anti-inflammatory, antioxidant, neuroprotectant, anxiolytic, antidepressant, analgesic, anti-tumor, and anti-psychotic properties. Moreover, delivery of CBD in a transdermal gel that has strong penetration enhancers and additional analgesic agents (e.g., menthol, eucalyptol, and salicin and other salicylic glycosides described below) results in greatly enhanced bioavailability and efficacy of the administered CBD. In a preferred aspect, about 1% by weight of CBD is added to the transdermal gel formulation. In other aspects, between 0.5% and 1.5%, or up to 3%, of CBD by weight may be added to the transdermal gel formulation.

In some aspects, 2,5-Dimethoxy-p-cymene is provided, typically as an extract of Arnica montana, to provide antifungal, antibacterial, and insecticidal properties useful in topical applications of the transdermal gel formulation. Furthermore, 2,5-Dimethoxy-p-cymene appears to act synergistically with conventional chemotherapy and radiotherapy, and suggests oncological benefits for the transdermal gel formulation.

In some aspects, extract of willow bark is included in the transdermal gel formulation to provide synergistic augmentation of the formulation's analgesic benefits. Specifically, an active ingredient of willow bark is salicin and other salicylic glycosides, which are closely related to salicylic acid (which is the well-known main ingredient of aspirin). Topical application of willow bark in an aspect of the invention provides excellent analgesic benefits both topically and systemically, without any involvement of the digestive system (where the side effects of aspirin can be significant). Since salicin and the other salicylic glycosides in willow bark are small molecules, they benefit from the synergistic penetration enhancement resulting from the use of eucalyptol and menthol for quicker skin penetration. Moreover, salicin and the other salicylic glycosides provide a synergistic analgesic effect with menthol, which has the effect dilating blood vessels in the area of gel application, thus increasing blood flow and the bioavailability od the salicylate glycosides to body tissues.

The skilled person will be aware of a range of possible modifications of the various aspects described above. Accordingly, the present invention is defined by the claims and their equivalents.

What is claimed is:

1. A composition for transdermal supplementation, comprising:
an aqueous gel comprising therapeutically effective amounts of:
carnosine;
aloe vera gel;
vitamin E; and
an extract of the Silybum marianum plant.

2. The composition of claim 1, wherein the extract of the Silybum marianum plant comprises silymarin.

3. The composition of claim 1, further comprising an additional therapeutically effective amount of a Lactobacillus ferment lysate.

4. A method of treatment using transdermal supplementation, comprising the step of:
applying an aqueous gel to a human's skin from a dispenser, the aqueous gel comprising:
carnosine;
aloe vera gel;
vitamin E; and
an extract of the Silybum marianum plant.

5. The method of treatment of claim 4, wherein the extract of the Silybum marianum plant comprises silymarin.

6. The method of treatment of claim 4, further comprising an additional therapeutically effective amount of a Lactobacillus ferment lysate.
